(19) United States
(12) Patent Application Publication
(10) Pub. No.: US 2023/0093889 A1
(43) Pub. Date: Mar. 30, 2023

(54) SIMULATED MOVING BED SEPARATION DEVICE AND METHOD WITH EXTENDED JET BREAKER

(71) Applicant: IFP Energies Nouvelles, Rueil-Malmaison (FR)

(72) Inventors: Guillaume BLANCKE, Rueil-Malmaison Cedex (FR); Amir Hossein AHMADI-MOTLAGH, Rueil-Malmaison Cedex (FR); Alexandre VONNER, Rueil-Malmaison Cedex (FR); Mohamed DAHRAOUI, Rueil-Malmaison (FR); Ayoub ABOURICHA, Rueil-Malmaison (FR)

(73) Assignee: IFP Energies Nouvelles, Rueil-Malmaison (FR)

(21) Appl. No.: 17/956,291

(22) Filed: Sep. 29, 2022

(30) Foreign Application Priority Data
Sep. 30, 2021 (FR) 2110-362
Sep. 30, 2021 (FR) 2110362

(51) Int. Cl. B01D 15/18; B01D 15/20; B01D 15/42

(52) U.S. Cl. CPC B01D 15/1842; B01D 15/1828; B01D 15/203; B01D 15/426; B01D 2215/024

(57) ABSTRACT

Distribution and collection panel comprising an upper screen (4), a collector (5), a separation plate (6) with outlet openings (11), a distributor (7), a lower screen (8), an injection/withdrawal tank (9) adjacent to the separation plate, and a jet breaker element (12) perpendicular to the flow (E) of a main fluid and comprising two solid jet breaker plates (13) that are: extended on either side of the injection/withdrawal tank; juxtaposed with the lower screen; disposed beneath the outlet openings (11); designed to direct the main fluid in the distributor in a direction orthogonal to the direction of the flow (E), the ratio I/L of the width I of the solid jet breaker plate to the width L of the lateral part of the separation plate being at least 0.1.

### TECHNICAL FIELD

The invention relates to the field of the separation of natural or chemical products that are difficult to separate by distillation. Use is then made of a family of methods, and of associated devices, known as simulated moving bed separation methods or devices, with either simulated countercurrent or simulated co-current, which will be referred to hereinafter as “SMB”.

The fields concerned are in particular the separation of para-xylene from other C8 aromatic isomers. Other fields concerned are, not exclusively:

the separation of, on the one hand, normal paraffins from, on the other hand, branched paraffins, naphthenes and aromatics; olefin/paraffin separation; the separation of meta-xylene from other C8 aromatic isomers; and the separation of ethylbenzene from other C8 aromatic isomers.

Specifically, the invention relates to an SMB device and method comprising a device for distributing and collecting fluids within a column implementing a flow of said fluids in a medium of solid particles, referred to as adsorbent bed or granular medium.

Column is understood to mean a column comprising a plurality of adsorbent beds disposed in series in a direction of flow of the one or more fluids implemented in the column. The fluid passing successively through the adsorbent beds is referred to as main fluid to distinguish it from other, secondary fluids that can be added to the main fluid via a distribution and collection device, also referred to as plate, generally situated between two successive beds.

A plate comprises at least one collection zone and a system of valves making it possible to collect main fluid and/or to inject secondary fluids and to mix these secondary fluids with the main fluid. A plate also comprises at least one distribution zone the aim of which is to distribute the fluid resulting from the mixing of the main fluid and the secondary fluids across the granular bed situated immediately downstream, in the direction of the flow of the main fluid. Reference will be made in the remainder of the text simply to “downstream” bed to denote the granular bed situated immediately downstream of the distributor according to the present invention.

The present invention relates to the distribution zone referred to below as distributor that makes it possible to feed each granular bed, or at least some of them, with a fluid that is in the form of a jet coming from the collection zone or from the system for mixing the main fluid and the secondary fluids with which is equipped the preceding granular bed, i.e. more specifically the “upstream” bed, in the direction of the flow of the main fluid.

### PRIOR ART

Numerous devices are known for distributing, mixing or collecting a fluid in a chamber containing solid particles, such as in particular a multistage column. The plates generally have the functions of distributing a fluid as homogenously as possible over the section of the column, of effectively mixing the main fluid passing through the various beds of the column with one or more secondary fluids introduced at each bed, optionally of collecting a flow of fluid between two beds, and finally of best homogenizing the concentrations at the bed outlet before the entry into the following bed of solid particles, i.e. the bed situated immediately downstream of the device in question.

In addition, the plates have to satisfy a certain number of constraints such as generating as little axial dispersion as possible, generating the minimum pressure drop, and not producing hydrodynamic disturbances that could have a negative effect on the performance of the method.

The plates have a certain number of features familiar to those skilled in the art.

For the sake of clarity of the text, a column is divided into a plurality of plates Pi and adsorbent beds Ai, the plate Pi being disposed directly upstream of the adsorbent bed Ai, in the direction of the flow of the main fluid. Furthermore, reference is made to adsorbent bed Ai+1 to denote the following adsorbent bed situated downstream of the adsorbent bed Ai, in the direction of the flow of the main fluid. In the same way, a plate Pi+1 denotes the following plate situated downstream of the plate Pi, in the direction of the flow of the main fluid.

Furthermore, each plate Pi of the column can have a plurality of systems of collection and injection valves and a plurality of distributors in connection with the way in which the plate can be divided into a plurality of sectors or regions, referred to as panels. Generally, each panel of the plate has one system of collection and injection valves and one distributor.

Each panel can have various shapes, the most common being the division into angular sectors or meridian panels, i.e. (mutually) parallel panels, substantially of the same width.

In each panel of a plate Pi, it is possible to:
collect main fluid from an adsorbent bed Ai−1 via a system referred to as collection baffle;
withdraw main fluid leaving the adsorbent bed Ai−1 or mix main fluid leaving the adsorbent bed Ai−1 with a secondary fluid optionally injected into the panel in question via a distribution network, ending in an injection/withdrawal tank;
redistributing the main fluid collected alone or as a mixture with a secondary fluid across the following adsorbent bed Ai via a distributor.

EP0074815, US2006/0108274A1, FR2708480 provide examples of plates used in the case of SMB adsorption.

In some cases, the bed of particles can be blocked by the distributor, i.e. there is no empty space between the distributor and the adsorbent bed Ai.

In the case in which there is an empty space between the distributor and the bed, as described in US2006/0108274A1, the distributor can be designed so as not to generate excessive fluid velocities locally at the bed inlet, so as not to cause partial fluidization of the bed of particles that could have a negative effect on the performance of the method. Specifically, US2006/0108274A1 describes a jet breaker plate positioned above the distributor, and beneath the open zones corresponding to the outlet for a jet of liquid, in order to limit the high velocities of this jet at the inlet of the downstream granular bed. By contrast, the distributor as described in US2006/0108274A1 may not be sufficient to eliminate the partial fluidization of the bed of particles, hence the need to resort to other solutions.

In order to further reduce the partial fluidization of the bed of particles, the prior art proposes multiple types of solutions:
positioning, downstream of the distributor, an element of screen or perforated plate type making it possible to limit the turbulence and high velocities at the inlet of the bed of particles;
increasing the number of panels and the opening of the collection baffles so as to decrease the velocity at which the fluid passes through the distribution/mixing device.

US2009/0321359A1 proposes a distributor comprising the following three elements, disposed from top to bottom in the direction of flow of the fluid:
a solid jet breaker situated approximately along the axis of the outlet opening of the collection baffle of the panel, and centred on the axis of said collection baffle;
an intermediate perforated plate extending laterally beyond the jet breaker, over a width between the width of said jet breaker and a higher value equal to half the width of the panel, to within plus or minus 5 cm, and with a degree of opening between 15% and 30%; and
a distribution plate extending over the entire section of the panel P, and with a degree of opening between 7% and 15%.

However, the hydrodynamics of the fluids inside the adsorbent beds can be improved.

### SUMMARY OF THE INVENTION

The problem that the present invention seeks to solve is that of improving the flow of the fluids inside a column having a multiplicity of adsorbent beds disposed in series in the direction of the flow of the fluids.

The present invention relates to a distribution and collection device, also referred to below as a panel, which makes it possible to collect the main fluid (fluid circulating in the column) from an upstream adsorbent bed and making it possible to feed a downstream adsorbent bed with main fluid. Advantageously, the distribution and collection device also has a system for mixing the main fluid with one or more secondary fluids.

According to a first aspect, the present invention can be defined as a device for distributing and collecting a main fluid, the device being designed to feed a downstream adsorbent bed of a simulated moving bed separation column, the device comprising at least one panel, said panel comprising, in the direction of the flow of the main fluid:
an upper screen designed to support a bed of solid particles of an upstream adsorbent bed;
a collector (or collection zone) designed to collect the main fluid leaving the upstream adsorbent bed;
a separation plate, separating the collector from a distributor and comprising at least one outlet opening for sending the main fluid from the collector towards the distributor (or distribution zone);
the distributor designed to distribute the main fluid across the downstream adsorbent bed; and
a lower screen, the panel also comprising:
an injection/withdrawal tank adjacent to the separation plate and disposed at a substantially central position of the panel, the separation plate comprising two lateral parts situated on either side of the injection/withdrawal tank, each lateral part extending over a width L from the injection/withdrawal tank to a lateral wall of the panel;
a jet breaker element extending perpendicular to the direction of the flow of the main fluid, the jet breaker element comprising two solid jet breaker plates that are:
   extended on either side of the injection/withdrawal tank;
   juxtaposed with the lower screen;
   disposed beneath the at least one outlet opening;
   designed to direct the main fluid in the distributor in a direction orthogonal to the direction of the flow of the main fluid,
      in which device the ratio I/L of the width I of each solid jet breaker plate to the width L of the lateral part of the separation plate is at least 0.1.

Advantageously, the jet breaker element makes it possible in particular:
to improve the flow by decreasing the axial dispersion of the fluid passing through the downstream adsorbent bed (best approaching plug flow),
to improve the hydrodynamics of the fluids inside the downstream adsorbent bed; and
to decrease the formation of furrows on the upper surface of the downstream adsorbent bed.

According to one or more embodiments, the jet breaker element comprises a central body disposed beneath the injection/withdrawal tank and connecting the two solid jet breaker plates.

According to one or more embodiments, the ratio I/L of the width I of the solid jet breaker plate to the width L of the lateral part of the separation plate is at least 0.2, preferably at least 0.25.

According to one or more embodiments, the ratio I/L of the width I of the solid jet breaker plate to the width L of the lateral part of the separation plate is between 0.1 and 0.7, preferably between 0.2 and 0.4, very preferably between 0.25 and 0.30.

According to one or more embodiments, the jet breaker element and the injection/withdrawal tank are juxtaposed.

According to one or more embodiments, the distance between the lower end of the separation plate and the upper end of the jet breaker element is less than 10%, and preferably less than 6%, of the width of the panel.

According to one or more embodiments, the separation plate has a degree of opening between 1% and 10%, and preferably between 4% and 8%.

According to one or more embodiments, the separation plate is perforated with holes 5 mm to 50 mm in diameter and/or 30 mm to 90 mm apart centre to centre.

According to a second aspect, the present invention can be defined as a distribution and collection plate of a simulated moving bed separation column, the plate comprising a plurality of devices according to the first aspect.

According to a third aspect, the present invention can be defined as a simulated moving bed separation column, comprising a plurality of plates according to the second aspect.

According to one or more embodiments, the column is divided into N adsorbent beds separated by n plates, the number of adsorbent beds N and the number of plates n being identical and between 4 and 24, and preferentially between 8 and 19, very preferentially between 12 and 15.

According to a fourth aspect, the present invention can be defined as a simulated moving bed separation unit comprising at least one column according to the third aspect.

According to a fifth aspect, the present invention can be defined as a simulated moving bed separation method, comprising the following steps: at least one column is fed with at least one feedstock and a desorbent, and at least one extract and at least one raffinate are withdrawn from the column, said column comprising one or more beds of an adsorbent solid that are interconnected in a closed loop and separated by plates comprising a plurality of devices according to the first aspect, the feed and withdrawal points in the plates of the column being shifted over time by a value corresponding to one adsorbent bed with a switching time and determining a plurality of operating zones of the column, and notably the following main zones:

by definition, each of the operating zones is denoted by a number:
zone I for desorption of a product to be separated (e.g. para-xylene) is between the injection of the desorbent and the withdrawal of the extract;
zone II for desorption of the isomers of the product to be separated is between the withdrawal of the extract and the injection of the feedstock;
zone III for adsorption of the product to be separated is between the injection of the feedstock and the withdrawal of the raffinate; and
zone IV is between the withdrawal of raffinate and the injection of desorbent; in which method the adsorbent beds are distributed in zones I to IV according to configurations referred to as a/b/c/d type configurations, i.e. the distribution of the beds is as follows:
a is the number of beds in zone I;
b is the number of beds in zone II;
c is the number of beds in zone III; and
d is the number of beds in zone IV.
   in which method:

a=(t*0.2)*(1±0.2);

b=(t*0.4)*(1±0.2);

c=(t*0.27)*(1±0.2); and

d=(t*0.13)*(1±0.2), or

a=(t*0.17)*(1±0.2);

b=(t*0.42)*(1±0.2);

c=(t*0.25)*(1±0.2); and

d=(t*0.17)*(1±0.2),

in which method t is a natural integer between 6 and 24, preferably between 8 and 19, very preferably between 12 and 15.

According to one or more embodiments:
the feedstock comprises a mixture of aromatics containing 8 carbon atoms; and/or
the desorbent is chosen from the group made up of one or more isomers of diethylbenzene and toluene, preferably the desorbent is para-diethylbenzene or toluene, very preferably the desorbent is toluene; and/or
the adsorbent used comprises or consists of a faujasite chosen from the group consisting of BaX, BaKX and BaLSX.
   According to one or more embodiments:
the temperature in the adsorbent beds is between 140° C. and 189° C., preferably between 155° C. and 185° C., very preferably between 170° C. and 180° C.; and/or
the pressure in the adsorbent beds is between 1 MPa and 10 MPa, preferably between 2 MPa and 4 MPa, very preferably between 2 MPa and 3 MPa; and/or
the switching time is between 30 seconds and 100 seconds, preferably between 40 seconds and 80 seconds; and/or
the surface velocity between the beds is between 0.2 cm/s and 2.5 cm/s and preferably between 0.5 cm/s and 2 cm/s.

Other features and advantages of the invention according to the aforementioned aspects will become apparent upon reading the following description and non-limiting exemplary embodiments, with reference to the appended figures described below.

### LIST OF FIGURES

FIG. 1 shows a view in partial cross section of a multistage column with three successive plates, each plate having a plurality of panels equipped with a collection system, a system for withdrawing the main fluid or injecting a secondary fluid and a reference distributor.

FIG. 2 shows a view in partial cross section of a multistage column with three successive plates, each plate having a plurality of panels equipped with a collection system, a system for withdrawing the main fluid or injecting a secondary fluid and a distributor according to the invention.

### DETAILED DESCRIPTION OF THE INVENTION

Embodiments of the device and of the method according to the aforementioned aspects will now be described in detail. In the following detailed description, numerous specific details are disclosed in order to provide a deeper understanding of the device and of the method. However, it will be apparent to those skilled in the art that the device and the method can be implemented without these specific details. In other cases, well-known features have not been described in detail in order to avoid unnecessarily complicating the description.

In the present application, the term “comprise” is synonymous with (means the same thing as) “include” and “contain”, and is inclusive or open and does not exclude other elements that are not stated. It is understood that the term “comprise” includes the exclusive and closed term “consist”. Furthermore, in the present description, the terms “essentially” or “substantially” correspond to an approximation of ±10%, preferably of ±5%, very preferably of ±2%. For example, an element disposed substantially at a certain position of a panel, can be disposed in the panel with an approximation of ±10%, preferably ±5%, with respect to the width or the height of the panel.

According to the first aspect, the invention can be defined as a distribution and collection device (also referred to below as a panel) for the SMB separation units, the distribution and collection device being designed to collect a fluid coming from an upstream adsorbent bed and distribute the fluid in the direction of a downstream adsorbent bed.

An SMB separation unit comprises at least one separation column divided into N adsorbent beds separated by n plates (defining inter-bed zones), it being possible for each plate to itself be divided into a plurality of panels. Preferably, the number of adsorbent beds N and the number of plates n are identical and are between 4 and 24, and preferentially between 8 and 19, very preferentially between 12 and 15.

The division of the plate Pi into panels is known from the prior art. The two most common types of division are division into meridian panels and division into panels corresponding to angular sectors. The meridian panels correspond to divisions of the plate Pi into elements that are mutually parallel and contiguous so as to ensure complete coverage of the horizontal section of the plate. The meridian panels are oriented according to a diameter of said plate, and preferably have substantially the same width. According to one or more embodiments, each plate is divided into between 4 and 24 panels, preferably between 12 and 16 panels. The panels are preferably meridian panels.

The Device

The distribution and collection device generally comprises, in the direction of the flow of the main fluid,
a collector designed to collect the main fluid leaving an upstream adsorbent bed;
an injection/withdrawal tank designed to extract the collected main fluid or inject a secondary fluid so as to mix said secondary fluid with the main fluid; and
a distributor designed to distribute the main fluid collected alone or as a mixture with a secondary fluid across the downstream adsorbent bed.

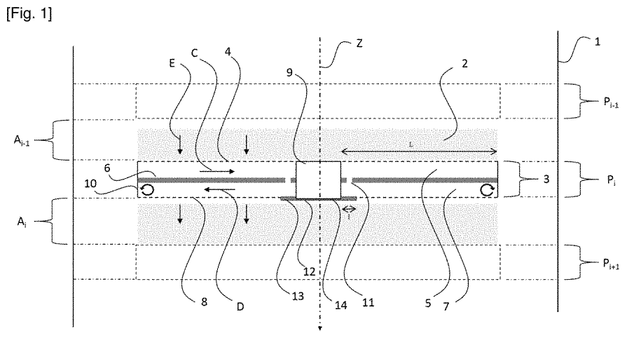

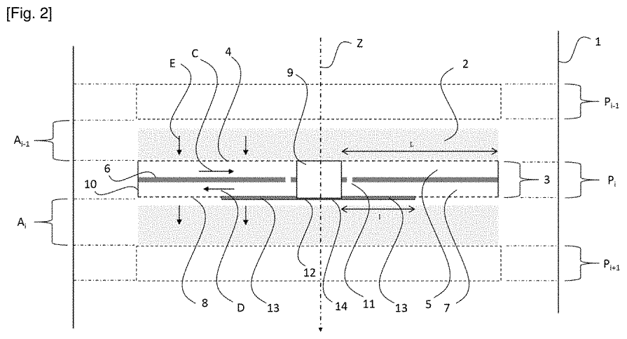

With reference to FIG. 1 and FIG. 2, a column 1 comprises a plurality of beds of solid particles 2 disposed in series in a direction of flow E of a main fluid implemented in the column 1. Specifically, the column has, in the direction of flow E of the main fluid: a plate Pi−1, an adsorbent bed Ai−1 (referred to as upstream adsorbent bed Ai−1), a plate Pi, an adsorbent bed Ai (referred to as downstream adsorbent bed Ai), and a plate Pi+1.

With reference to FIG. 1 and FIG. 2, the plate Pi is divided into a plurality of panels 3, each panel 3 preferably comprising vertical walls, including two lateral walls 10, each panel 3 also comprising, in the direction of the flow E of the main fluid:
an upper screen 4 or any other equivalent device (e.g. perforated plate) making it possible to support the bed of solid particles 2;
a collector 5 or collection zone (also depicted by the collection channel C in the figures) designed to collect the main fluid leaving the upstream adsorbent bed Ai−1;
a separation plate 6, separating the collector 5 from the distributor 7;
the distributor 7 or distribution zone (also depicted by the distribution channel D in the figures) designed to distribute the main fluid, collected alone or as a mixture with a secondary fluid, across the downstream adsorbent bed Ai;
a lower screen 8 or any other equivalent device (e.g. perforated plate) making it possible to support the panel 3;
the upper screen 4, the collector 5, the separation plate 6, the distributor 7 and the lower screen 8 extending from one lateral wall 10 to the other lateral wall 10.

With reference to FIG. 1 and FIG. 2, the panel 3 also comprises an injection/withdrawal tank 9 designed to extract main fluid collected by the collector 5 or inject a secondary fluid so as to mix said secondary fluid with the main fluid. The injection/withdrawal tank 9 is adjacent to the separation plate 6 and is disposed at a central position of the panel 3, i.e. situated substantially along the central axis Z of the panel 3 (as shown in the view in cross section in FIGS. 1 and 2). The central axis Z of the panel 3 is a transverse axis of the panel 3, i.e. an axis parallel to the direction of the flow E and orthogonal to the plane formed by the separation plate 6.

Advantageously, the upper screen 4 and the separation plate 6 together form the collector 5 (collection zone) designed to direct the main fluid towards the injection/withdrawal tank 9.

Advantageously, the separation plate 6 comprises two lateral parts situated on either side of the injection/withdrawal tank 9, i.e. the injection/withdrawal tank 9 separates the separation plate 6 into two lateral parts, each lateral part extending over a width L from the injection/withdrawal tank 9 to a lateral wall 10 of the panel 3, respectively. It is understood that the width of the panel is equal to the distance between the two lateral walls 10, i.e. the width of the panel is equal to the sum of the width of the injection/withdrawal tank 9 and the widths L of the two lateral parts situated on either side of the injection/withdrawal tank 9.

In the detailed description, well-known features of the injection/withdrawal tank 9 have not been described in detail in order to avoid unnecessarily complicating the description. For example, with reference to FIG. 1 and FIG. 2, the injection/withdrawal tank 9 extends substantially from the upper screen 4 to the lower screen 8. However, it will be apparent to those skilled in the art that the injection/withdrawal tank 9 can extend substantially from the upper screen 4 to the separation plate 6, or from the separation plate 6 to the lower screen 8.

It is also understood that the injection/withdrawal tank 9 can be disposed between the collector 5 and the separation plate 6, or between the separation plate 6 and the distributor 7, or between the collector 5 and the distributor 7.

Advantageously, the separation plate 6 comprises at least one, and preferably at least two, outlet openings 11, preferably disposed near the injection/withdrawal tank 9 and designed to send the main fluid from the collector towards the distributor 7. Preferably, at least one outlet opening 11 is disposed on either side of the injection/withdrawal tank 9. Depending on the operating mode of the panel 3, the main fluid can thus be collected in the injection/withdrawal tank 9 or mixed with a secondary fluid leaving the injection/withdrawal tank 9. The main fluid and the secondary fluid that are thus mixed are redistributed towards the downstream adsorbent bed Ai by passing through the distributor 7. According to one or more embodiments, the term near corresponds to a distance less than 10%, preferably less than 5%, of the width L of the lateral parts of the separation plate 6.

Advantageously, the lower screen 8 and the separation plate 6 together form the distributor 7 (distribution zone) for directing the main fluid collected alone or as a mixture with a secondary fluid towards the downstream adsorbent bed Ai.

With reference to FIG. 1 and FIG. 2, the panel 3 also comprises a jet breaker element 12 extending perpendicular to the direction of the flow E of the main fluid and disposed beneath the at least one outlet opening 11. Advantageously, the jet breaker element 12 is designed to direct the main fluid, alone or as a mixture with the secondary fluid, in the distributor 7 in a direction orthogonal to the direction of the flow E of the main fluid, i.e. towards the lateral wall 10 of the panel 3.

Advantageously, the jet breaker element 12 and the lower screen 8 adjoin one another in the sense that they are juxtaposed with one another (e.g. screwed, welded, riveted, adhesively bonded etc. to one another).

Advantageously, the jet breaker element 12 is disposed beneath the separation plate 6 and beneath the injection/withdrawal tank 9, and is disposed at a substantially central position of the panel 3, i.e. situated substantially along the central axis Z of the panel 3.

With reference to FIG. 1 and FIG. 2, the jet breaker element 12 comprises two solid jet breaker plates 13 of width I and extending on either side of (and from) the injection/withdrawal tank 9 towards the lateral wall 10 of the panel 3. According to one or more embodiments, the jet breaker element 12 also comprises an optional central body 14 disposed beneath the injection/withdrawal tank 9, connecting the two solid jet breaker plates 13.

With reference to FIG. 1, the ratio I/L of the width I of each reference solid jet breaker plate 13 to the width L of a lateral part of the separation plate 6 is less than 0.05.

With reference to FIG. 2, the ratio I/L of the width I of each solid jet breaker plate 13 according to the invention to the width L of a lateral part of the separation plate 6 is at least 0.1, preferably at least 0.2, very preferably at least 0.25. According to one or more embodiments, the ratio I/L is between 0.1 and 0.7, preferably between 0.2 and 0.4, very preferably between 0.25 and 0.30.

According to one or more embodiments, the jet breaker element 12 extends over a total width of at least 10% of the width of the panel 3, preferably at least 20% of the width of the panel 3, very preferably at least 25% of the width of the panel 3. According to one or more embodiments, the jet breaker element 12 extends over a total width of between 10% and 70%, preferably between 20% and 40%, very preferably between 25% and 30%, of the width of the panel 3.

According to one or more embodiments, the jet breaker element 12 and the injection/withdrawal tank 9 adjoin one another in the sense that they are juxtaposed with one another. According to one or more embodiments, the jet breaker element 12, the lower screen and the injection/withdrawal tank 9 adjoin one another in the sense that they are juxtaposed with one another.

According to one or more embodiments, the lower screen is a screen, for example, of the “Johnson” type (slots being substantially perpendicular to the central axis of the panel). According to one or more embodiments, the lower screen 8 is a perforated plate.

According to one or more embodiments, the distance between the lower end of the separation plate 6 separating the distributor 7 from the collector 5 and the upper end of the jet breaker element 12 is less than 10%, and preferably less than 6%, of the width of the panel 3.

According to one or more embodiments, the distance between the lower end of the separation plate 6 separating the distributor 7 from the collector 5 and the upper end of the jet breaker element 12 is between 1 mm and 50 mm, and preferentially between 5 mm and 30 mm.

According to one or more embodiments, the separation plate 6 has a degree of opening between 1% and 10%, and preferentially between 4% and 8%.

According to one or more embodiments, the separation plate 6 is perforated with holes 5 mm to 50 mm in diameter and/or 30 mm to 90 mm apart centre to centre.

The invention also relates to a plate Pi comprising a plurality of panels 3 according to the invention.

The invention also relates to a separation column 1 divided into N adsorbent beds Ai separated by n plates Pi comprising a plurality of panels 3 according to the invention.

The invention also relates to an SMB separation unit comprising at least one separation column 1 divided into N adsorbent beds Ai separated by n plates Pi comprising a plurality of panels 3 according to the invention.

The Method

The invention can also be defined as an SMB method involving an SMB separation unit according to the invention, in which the feedstock to be separated is any mixture of compounds, such as aromatics having from 7 to 9 carbon atoms, a mixture of normal and iso-paraffins, or a mixture of normal and iso-olefins.

Thus, the invention also relates to an SMB separation method using at least one separation column 1 divided into N adsorbent beds Ai separated by n plates Pi comprising a plurality of panels 3 according to the invention.

In the remainder of the text, reference is made to step to denote an operation or a group of similar operations carried out on a given stream at a certain point of the method. The method is described in its various steps taken in the order of flow of the streams or products.

The SMB separation method comprises the following steps: the column 1 is fed with at least one feedstock and a desorbent, and at least one extract and at least one raffinate are withdrawn from the column 1, said column 1 comprising one or more beds of an adsorbent solid Ai that are interconnected in a closed loop (i.e. the last bed of the last adsorber being designed to send the circulating stream to the first bed of the first adsorber) and separated by plates Pi according to the invention, the feed and withdrawal points in the plates of the column being shifted over time by a value corresponding to one adsorbent bed with a switching time (denoted ST) and determining a plurality of operating zones of the SMB device, and notably the following main zones, denoted by definition by a number:
zone I for desorption of the product (of interest) to be separated is between the injection of the desorbent and the withdrawal of the extract;
zone II for desorption of the isomers of the product to be separated is between the withdrawal of the extract and the injection of the feedstock;
zone III for adsorption of the product to be separated is between the injection of the feedstock and the withdrawal of the raffinate; and
zone IV is between the withdrawal of raffinate and the injection of desorbent.

According to one or more embodiments, the adsorbent beds are distributed in zones I to IV according to configurations referred to as a/b/c/d type configurations, i.e. the distribution of the beds is as follows:
a is the number of beds in zone I;
b is the number of beds in zone II;
c is the number of beds in zone III; and
d is the number of beds in zone IV.
   According to one or more embodiments:

a=(t*0.2)*(1±0.2);

b=(t*0.4)*(1±0.2);

c=(t*0.27)*(1±0.2); and

d=(t*0.13)*(1±0.2), and

in which t is a natural integer between 6 and 24, preferably between 8 and 19 (e.g. between 12 and 15).

According to one or more embodiments:

a=(t*0.17)*(1±0.2);

b=(t*0.42)*(1±0.2);

c=(t*0.25)*(1±0.2); and

d=(t*0.17)*(1±0.2), and

in which t is a natural integer between 6 and 24, preferably between 8 and 19, very preferably between 12 and 15 (e.g. 12 or 15).

According to one or more embodiments, the desorbent is chosen from the group made up of one or more isomers of diethylbenzene and toluene. According to one or more embodiments, the desorbent is para-diethylbenzene or toluene. According to one or more embodiments, the desorbent is toluene.

According to one or more embodiments, the adsorbent used comprises/consists of a faujasite chosen from the group consisting of BaX, BaKX and BaLSX.

According to one or more embodiments, the feedstock is a mixture of essentially C8 aromatic compounds (e.g. xylenes and ethylbenzene). According to one or more embodiments, the mixture comprises at least 95%, preferably at least 97% (e.g. at least 99%) of essentially C8 aromatic compounds. According to one or more embodiments, the feedstock comprises at least 15 wt % of para-xylene and/or 30 wt % of meta-xylene relative to the total weight of the feedstock.

One example of an SMB separation method of great industrial importance is the separation of C8 aromatic fractions in order to produce para-xylene of commercial purity, typically at a purity of at least 99.7 wt %, and a raffinate rich in ethylbenzene, ortho-xylene and meta-xylene.

The extract produced contains desorbent, para-xylene and optionally traces of isomers (para-xylene purity of greater than 95%, preferably greater than 98%). This extract can be treated in order to separate the desorbent (e.g. by distillation) and then purified either by crystallization or by SMB adsorption in order to increase the purity of the para-xylene.

According to one or more embodiments, the temperature in the adsorbent beds is between 140° C. and 189° C. and preferably between 155° C. and 185° C., particularly preferably between 170° C. and 180° C.

The pressure is adjusted so that the liquid phase is maintained at all points of the method according to the invention. According to one or more embodiments, the pressure in the adsorbent beds is between 1 MPa and 10 MPa, preferably between 2 MPa and 4 MPa, preferably between 2 MPa and 3 MPa.

According to one or more embodiments, the switching time ST (time between two successive switchings of the feeds/extractions) used is between 30 seconds and 100 seconds. Preferably, the switching time ST used is between 40 seconds and 80 seconds (e.g. 60±10 seconds).

According to one or more embodiments, the surface velocity between the beds is between 0.2 cm/s and 2.5 cm/s and preferentially between 0.5 cm/s and 2 cm/s.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding application No. FR 2110362, filed Sep. 30, 2021 are incorporated by reference herein.

### EXAMPLES

The effectiveness of the device according to the invention was tested by simulation. A first simulation, denoted example 1, reproduces a reference plate Pi as shown in FIG. 1 feeding an adsorbent bed Ai.

A second simulation, denoted example 2, reproduces a plate Pi according to the invention and as shown in FIG. 2 feeding an adsorbent bed Ai.

In the two examples, the structural features of the elements of the plate Pi and of the adsorbent bed Ai are as follows:
the diameter of the plate Pi is 9.7 m;
the plate is divided into meridian panels;
the width of the meridian panel is 1.15 m, the height of a bed is 1.25 m;
the separation plate 6 is perforated;
the jet breaker element 12 and the lower screen 8 are welded to one another;
the lower screen 8 is perforated;
all the elements are centred with respect to the injection/withdrawal tank 9;
the adsorbent bed Ai is filled with a zeolite sieve with a particle size centred around 550 μm;
the adsorbent bed is filled up to a height of 30 mm beneath the distributor 7;
the adsorbent bed Ai is fed with liquid at the surface velocity of 1.0 cm/s;

In the reference example 1, the width I of the reference solid jet breaker plates 13 is 3 cm.

In example 2 according to the invention, the width I of the solid jet breaker plates 13 according to the invention is 15 cm.

The results are shown in the form of the Péclet number Pe, which expresses the ratio between the convection of the fluid and the dispersion by axial diffusion. The higher the Péclet number, the lower the axial dispersion.

### Reference Example 1: Pe=120

### Example 2 According to the Invention: Pe=170

Minimizing the axial dispersion is beneficial to methods implementing fixed beds of solid particles, including adsorption methods in particular.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Device for distributing and collecting a main fluid, the device being designed to feed a downstream adsorbent bed (Ai) of a simulated moving bed separation column (1), the device comprising at least one panel (3), said panel (3) comprising, in the direction of the flow (E) of the main fluid:
an upper screen (4) designed to support a bed of solid particles (2) of an upstream adsorbent bed (Ai−1);
a collector (5) designed to collect the main fluid leaving the upstream adsorbent bed (Ai−1);
a separation plate (6), separating the collector (5) from a distributor (7) and comprising at least one outlet opening (11) for sending the main fluid from the collector (5) towards the distributor (7);
the distributor (7) designed to distribute the main fluid across the downstream adsorbent bed (Ai); and
a lower screen (8),
the panel also comprising:
an injection/withdrawal tank (9) adjacent to the separation plate (6) and disposed at a substantially central position of the panel (3), the separation plate (6) comprising two lateral parts situated on either side of the injection/withdrawal tank (9), each lateral part extending over a width L from the injection/withdrawal tank (9) to a lateral wall (10) of the panel (3);
a jet breaker element (12) extending perpendicular to the direction of the flow (E) of the main fluid, the jet breaker element (12) comprising two solid jet breaker plates (13) that are:
extended on either side of the injection/withdrawal tank (9);
juxtaposed with the lower screen (8);
disposed beneath the at least one outlet opening (11);
designed to direct the main fluid in the distributor (7) in a direction orthogonal to the direction of the flow (E) of the main fluid,
in which panel the ratio I/L of the width I of each solid jet breaker plate (13) to the width L of the lateral part of the separation plate (6) is at least 0.1.

2. Device according to claim 1, wherein the jet breaker element (12) comprises a central body (14) disposed beneath the injection/withdrawal tank (9) and connecting the two solid jet breaker plates (13).

3. Device according to claim 1, wherein the ratio I/L of the width I of the solid jet breaker plate (13) to the width L of the lateral part of the separation plate (6) is at least 0.2, preferably at least 0.25.

4. Device according to claim 1, wherein the ratio I/L of the width I of the solid jet breaker plate (13) to the width L of the lateral part of the separation plate (6) is between 0.1 and 0.7, preferably between 0.2 and 0.4, very preferably between 0.25 and 0.30.

5. Device according to claim 1, wherein the jet breaker element (12) and the injection/withdrawal tank (9) are juxtaposed.

6. Device according to claim 1, wherein the distance between the lower end of the separation plate (6) and the upper end of the jet breaker element (12) is less than 10%, and preferably less than 6%, of the width of the panel (3).

7. Device according to claim 1, wherein the separation plate (6) has a degree of opening between 1% and 10%, and preferably between 4% and 8%.

8. Device according to claim 1, wherein the separation plate (6) is perforated with holes 5 mm to 50 mm in diameter and/or 30 mm to 90 mm apart centre to centre.

9. Distribution and collection plate (Pi) of a simulated moving bed separation column (1), the plate (Pi) comprising a plurality of devices according to claim 1.

10. Simulated moving bed separation column (1), comprising a plurality of plates (Pi) according to claim 9.

11. Column (1) according to claim 10, divided into N adsorbent beds (Ai) separated by n plates (Pi), the number of adsorbent beds N and the number of plates n being identical and being between 4 and 24, and preferentially between 8 and 19, very preferentially between 12 and 15.

12. Simulated moving bed separation unit comprising at least one column (1) according to claim 10.

13. Simulated moving bed separation method, comprising the following steps: at least one column (1) is fed with at least one feedstock and a desorbent, and at least one extract and at least one raffinate are withdrawn from the column (1), said column (1) comprising one or more beds of an adsorbent solid (Ai) that are interconnected in a closed loop and separated by plates (Pi) comprising a plurality of devices according to claim 1, the feed and withdrawal points in the plates (Pi) of the column (1) being shifted over time by a value corresponding to one adsorbent bed with a switching time and determining a plurality of operating zones of the column (1), and notably the following main zones denoted by definition by a number:
zone I for desorption of a product to be separated is between the injection of the desorbent and the withdrawal of the extract;
zone II for desorption of the isomers of the product to be separated is between the withdrawal of the extract and the injection of the feedstock;
zone III for adsorption of the product to be separated is between the injection of the feedstock and the withdrawal of the raffinate; and
zone IV is between the withdrawal of raffinate and the injection of desorbent;
in which method the adsorbent beds are distributed in zones I to IV according to configurations referred to as a/b/c/d type configurations, i.e. the distribution of the beds is as follows:
a is the number of beds in zone I;
b is the number of beds in zone II;
c is the number of beds in zone III; and
d is the number of beds in zone IV.
in which method:
a=(t*0.2)*(1±0.2);
b=(t*0.4)*(1±0.2);
c=(t*0.27)*(1±0.2); and
d=(t*0.13)*(1±0.2), or
a=(t*0.17)*(1±0.2);
b=(t*0.42)*(1±0.2);
c=(t*0.25)*(1±0.2); and
d=(t*0.17)*(1±0.2),
in which method t is a natural integer between 6 and 24, preferably between 8 and 19, very preferably between 12 and 15.

14. Method according to claim 13, comprising at least one of the following operating conditions:
the feedstock comprises a mixture of aromatics containing 8 carbon atoms;
the desorbent is chosen from the group made up of one or more isomers of diethylbenzene and toluene, preferably the desorbent is para-diethylbenzene or toluene, very preferably the desorbent is toluene;
the adsorbent used comprises or consists of a faujasite chosen from the group consisting of BaX, BaKX and BaLSX.

15. Method according to claim 13, comprising at least one of the following operating conditions:
the temperature in the adsorbent beds is between 140° C. and 189° C., preferably between 155° C. and 185° C., very preferably between 170° C. and 180° C.;
the pressure in the adsorbent beds is between 1 MPa and 10 MPa, preferably between 2 MPa and 4 MPa, very preferably between 2 MPa and 3 MPa;
the switching time is between 30 seconds and 100 seconds, preferably between 40 seconds and 80 seconds;
the surface velocity between the beds is between 0.2 and 2.5 cm/s and preferably between 0.5 and 2 cm/s.
